(19) United States
(12) Patent Application Publication
(10) Pub. No.: US 2016/0143834 A1
(43) Pub. Date: May 26, 2016

(54) HAIR CARE COMPOSITIONS COMPRISING DEHYDROXANTHAN GUM

(71) Applicant: The Procter & Gamble Company, Cincinnati, OH (US)

(72) Inventors: Kelly Rose KROGER LYONS, LIberty Twp., OH (US); Kendrick Jon HUGHES, Cincinnati, OH (US)

(21) Appl. No.: 14/946,042

(22) Filed: Nov. 19, 2015

Related U.S. Application Data
(60) Provisional application No. 62/082,384, filed on Nov. 20, 2014.

(51) Int. Cl. A61K 8/73; A61Q 5/06; A61K 8/49; A61K 8/42; A61K 8/34

(52) U.S. Cl. CPC A61K 8/73; A61K 8/42; A61K 8/34; A61K 8/4953; A61K 8/4926; A61Q 5/06; A61K 2800/592

(57) ABSTRACT

Provided is a hair care composition for providing improved root lift including at least 0.1% dehydroxanthan gum, from about 0.2% to about 1% of a panthenol compound, from about 0.1% to about 10% of a rheology modifier, and a carrier.

### FIELD OF THE INVENTION

Described herein are hair care compositions comprising dehydroxanthan gum and methods of use thereof.

### BACKGROUND OF THE INVENTION

Many attributes contribute to the appearance of hair considered to be attractive. For instance, hair with a full, thick, and voluminous appearance is very desirable. In contrast, hair with a thin and flat appearance is not as attractive, and can even lead to a perception that the thin-haired individual is older than his or her chronological age. Because of the foregoing problems associated with thin and flat hair, many individuals expend great effort and time on grooming, yet still do not attain their desired hairstyle and appearance. This can lead to frustration and/or lack of confidence in his or her appearance. These problems can be experienced by both female and male consumers and at a variety of ages. One way of addressing the appearance of thin and/or flat hair is by applying a root lift composition to the hair.

In view thereof, there is a need to provide consumers with an improved hair care composition that lifts the roots of the hair and therefore creates a fuller and thicker appearance of the hair.

### SUMMARY OF THE INVENTION

Provided is a hair care composition for providing improved root lift comprising (a) from about 0.3% to about 1% dehydroxanthan gum, by weight of the hair care composition; (b) from about 0.2% to about 1% of a panthenol compound, by weight of the hair care composition; (c) from about 0.1% to about 10% of a rheology modifier, by weight of the hair care composition; and (d) a carrier.

Also provided is a hair care composition for providing improved root lift comprising (a) from about 0.3% to about 1% dehydroxanthan gum, by weight of the hair care composition; (b) from about 0.2% to about 1% of a panthenol compound, by weight of the hair care composition; (c) from about 0.1% to about 10% of a rheology modifier, by weight of the hair care composition; (d) from about 25% to about 50% alcohol, by weight of the hair care composition; (e) from about 0.01% to about 1% caffeine, by weight of the hair care composition; (f) from about 0.01% to about 4% niacinimide, by weight of the hair care composition; and (g) water.

These and other features, aspects, and advantages of the present invention will become evident to those of ordinary skill in the art from a reading of the present disclosure.

### BRIEF DESCRIPTION OF THE DRAWINGS

While the specification concludes with the claims particularly pointing out and distinctly claiming the invention, it is believed that the present invention will be better understood from the following description taken in conjunction with the accompanying drawings in which:

FIG. 1A is a switch of hair with measurements relevant to the Root Lift Test Method;

FIG. 1B is a switch of hair with measurements relevant to the Root Lift Test Method;

FIG. 1C is a switch of hair with measurements relevant to the Root Lift Test Method; and

FIG. 2 is a graph of the “2 hr Average Root Lift Results” from Table 4.

The patent or application file contains at least one photograph executed in color. Copies of this patent or patent application publication with color photographs will be provided by the Office upon request and payment of the necessary fee.

### DETAILED DESCRIPTION OF EMBODIMENTS OF THE INVENTION

While the specification concludes with the claims particularly pointing and distinctly claiming the invention, it is believed that embodiments of the present invention will be better understood from the following description. In all embodiments of the present invention, all weight percentages are by weight of the total composition, unless specifically stated otherwise. All ratios are weight ratios, unless specifically stated otherwise. All ranges are inclusive and combinable. The number of significant digits conveys neither limitations on the indicated amounts nor on the accuracy of the measurements. All numerical amounts are understood to be modified by the word “about” unless otherwise specifically indicated. All measurements are understood to be made at 25° C. and at ambient conditions, where “ambient conditions” means conditions under about one atmosphere of pressure and at about 50% relative humidity. All such weights as they pertain to listed ingredients are based on the active level and do not include carriers or by-products that may be included in commercially available materials, unless otherwise specified.

As used herein, the term “hair care composition” means a composition that is applied to the hair and/or the skin underneath the hair, including compositions used to treat or care for the hair. Products contemplated by the phrase “hair care composition” include, but are not limited to after-shave tonics and lotions, creams, emulsions, foams, hair conditioners (rinse-off and leave-on), hair colorants, hair tonics, liquids, lotions, mousses, propellant lotions, shampoos, shave gels, temporary beard hair dyes, and the like.

As used herein, the term “hair” may be “living” i.e. on a living body or may be “non-living” i.e. in a wig, hairpiece or other aggregation of non-living keratinous fibers. However, wool, fur, and other keratin containing fibers may be suitable substrates for the hair care compositions described herein.

The hair care composition for providing improved root lift described herein comprises (a) from about 0.3% to about 1% dehydroxanthan gum, by weight of the hair care composition; (b) from about 0.2% to about 1% of a panthenol compound, by weight of the hair care composition; and (c) from about 0.1% to about 10% of a rheology modifier, by weight of the hair care composition.

### I. Dehydroxanthum Gum

The hair care composition described herein comprises at least 0.1%, alternatively at least 0.6%, alternatively at least 0.65%, alternatively at least 0.7%, alternatively at least 0.75%, alternatively from about 0.1% to about 1%, alternatively from about 0.6% to about 1%, alternatively from about 0.65% to about 1%, alternatively from about 0.6% to about 0.9%, alternatively from about 0.65% to about 0.95%, alternatively from about 0.7% to about 1%, and alternatively about 0.75% dehydroxanthan gum, by weight of the hair care composition.

Exemplary dehydroxanthan gum may be obtained from AkzoNobel under the trade name AMAZE XT. In an embodiment, the dehydroxanthan gum may be formulated with cationic polymers. It may be used with other styling polymers such as acrylate polymers, polyurethane polymers, as well as with PVP and PVP/VA. In an embodiment, the dehydroxanthan gum may be combined with additional thickeners.

### II. Panthenol Compound

The hair care composition described herein comprises from about 0.2% to about 1%, alternatively from about 0.3% to about 0.8%, and alternatively from about 0.4% to about 0.8% panthenol.

As used herein, the term “panthenol compound” includes panthenol, one or more pantothenic acid derivatives, and mixtures thereof. Non-limiting examples of panthenol compounds include D-panthenol ([R]-2,4-dihydroxy-N-[3-hydroxypropyl)]-3,3-dimethylbutamide), D,L-panthenol, pantothenic acids and their salts (e.g., the calcium salt), panthenyl triacetate, royal jelly, panthetine, pantotheine, panthenyl ethyl ether, pangamic acid, pantoyl lactose, Vitamin B complex, or mixtures thereof.

### III. Rheology Modifier

The hair care composition described herein comprises a rheology modifier to increase the substantivity of the composition. Any suitable rheology modifier can be used. In an embodiment, the hair care composition may comprise from about 0.1% to about 10% of a rheology modifier, alternatively from about 0.5% to about 2.2% of a rheology modifier, alternatively from about 0.7% to about 2% of a rheology modifier, alternatively from about 1% to about 1.7% of a rheology, and alternatively from about 1% to about 1.5% of a rheology modifier, by weight of the hair care composition. In an embodiment, the rheology modifier may be a polyacrylamide thickener.

Non-limiting examples of rheology modifiers include acrylamide/ammonium acrylate copolymer (and) polyisobutene (and) polysorbate 20; acrylamide/sodium acryloyldimethyl taurate copolymer/isohexadecane/polysorbate 80; acrylates copolymer; acrylates/beheneth-25 methacrylate copolymer; acrylates/C10-C30 alkyl acrylate crosspolymer; acrylates/steareth-20 itaconate copolymer; ammonium polyacrylate/Isohexadecane/PEG-40 castor oil; C12-16 alkyl PEG-hydroxypropylhydroxyethyl ethylcellulose (HM-EHEC); carbomer; crosslinked polyvinylpyrrolidone (PVP); dibenzylidene sorbitol; hydroxyethyl ethylcellulose (EHEC); hydroxypropyl methylcellulose (HPMC); hydroxypropyl methylcellulose (HPMC); hydroxypropylcellulose (HPC); methylcellulose (MC); methylhydroxyethyl cellulose (MEHEC); PEG-150/decyl alcohol/SMDI copolymer; PEG-150/stearyl alcohol/SMDI copolymer; polyacrylamide/C13-14 isoparaffin/laureth-7; polyacrylate 13/polyisobutene/polysorbate 20; polyacrylate crosspolymer-6; polyamide-3; polyquaternium-37 (and) hydrogenated polydecene (and) trideceth-6; polyurethane-39; sodium acrylate/acryloyldimethyltaurate/dimethylacrylamide; crosspolymer (and) isohexadecane (and) polysorbate 60; sodium polyacrylate. Exemplary commercially-available rheology modifiers include ACULYN™ 28, Klucel M CS, Klucel H CS, Klucel G CS, SYLVACLEAR AF1900V, SYLVACLEAR PA1200V, Benecel E10M, Benecel K35M, Optasense RMC70, ACULYN™33, ACULYN™46, ACULYN™22, ACULYN™44, Carbopol Ultrez 20, Carbopol Ultrez 21, Carbopol Ultrez 10, Carbopol 1342, Sepigel™ 305, Simulgel™600, Sepimax Zen, and combinations thereof.

### IV. Carrier

According to another aspect of the present invention, the hair care compositions may further include at least 20% of one or more aqueous carriers, by weight of the hair care composition. According to an embodiment, the aqueous carrier may be prepared from demineralized or distilled water. Other acceptable carriers that may be used in the aqueous carrier include, but are not limited to, alcohol compounds, such as ethanol. According to an embodiment, the composition comprises alcohol, dipropylene glycol, and/or water.

The hair care composition described herein may have a pH ranging from about 3.0 to about 10, which may be measured by taking a direct pH measurement using a standard hydrogen electrode of the composition at 25° C. Accordingly, the pH of the hair care composition may be within the range from about 6 to about 9, for example.

### VI. Optional Ingredients

The hair care composition described herein may further comprise any suitable optional ingredients as desired. For example, the composition can optionally include other active or inactive ingredients.

The hair care composition described herein may include other common hair ingredients such as pyrithione zinc, minoxidil, silicones, glycerin, conditioning agents, and other suitable materials. The CTFA Cosmetic Ingredient Handbook, Tenth Edition (published by the Cosmetic, Toiletry, and Fragrance Association, Inc., Washington, D.C.) (2004) (hereinafter “CTFA”), describes a wide variety of nonlimiting materials that can be added to the composition herein. Examples of these ingredient classes include, but are not limited to: abrasives, absorbents, aesthetic components such as fragrances, pigments, colorings/colorants, essential oils, skin sensates, astringents, etc. (e.g., clove oil, menthol, camphor, eucalyptus oil, eugenol, menthyl lactate, witch hazel distillate), anti-acne agents, anti-caking agents, antifoaming agents, antimicrobial agents (e.g., iodopropyl butylcarbamate), antioxidants, binders, biological additives, buffering agents, bulking agents, chelating agents, chemical additives, colorants, cosmetic astringents, cosmetic biocides, denaturants, drug astringents, external analgesics, film formers or materials, e.g., polymers, for aiding the film-forming properties and substantivity of the composition (e.g., copolymer of eicosene and vinyl pyrrolidone), opacifying agents, pH adjusters, propellants, reducing agents, sequestrants, rheology modifiers, hair conditioning agents, and surfactants.

The hair care composition described herein may be in a form selected from the group consisting of solutions, dispersion, emulsions, powders, talcs, encapsulated, spheres, spongers, solid dosage forms, foams, and other delivery mechanisms. The hair care composition descried herein may also be selected from the group consisting of hair tonics, leave-on hair products such as conditioners, treatment, and styling products, rinse-off hair products such as conditioners, shampoos, and treatment products, and any other form that may be applied to the hair, in an embodiment, the roots of the hair.

### Vitamin B₃ Compounds

The hair care composition described herein may further include a vitamin B3 compound. As used herein, “vitamin B₃ compound” means nicotinic acid, niacinamide, nicotinyl alcohol, derivatives thereof, and mixtures thereof. The vitamin B₃ compound may be included as the substantially pure material, or as an extract obtained by suitable physical and/or chemical isolation from natural (e.g., plant) sources. Accordingly, the composition may include from about 0.1% to about 6% of the vitamin B₃ compound, alternatively from about 0.01% to about 4% of the vitamin B₃ compound, alternatively from about 1% to about 4% of the vitamin B₃ compound, and alternatively from about 2.5% to about 3.5% of the vitamin B₃ compound, wherein the percentage is a weight percentage based on the total weight of the final hair care composition.

In an embodiment, the vitamin B₃ compound may be naicinimide.

### Caffeine

In an embodiment, the hair care composition described herein may comprise from about 0.01% to about 1%, alternatively from about 0.65% to about 0.85%, alternatively from about 0.7% to about 0.8% caffeine, by weight of the hair care composition. In an embodiment, the caffeine included in the composition may be hydrous caffeine, caffeine salts, or complexes breaking down to yield caffeine.

### VII. Method for Providing Hair Lift

The hair care composition described herein may be used in a method for providing hair lift. The method may comprise applying the hair care composition as described herein to a region of the hair.

### Examples

The following are non-limiting examples of the hair care composition described herein. The examples are given solely for the purpose of illustration and are not to be construed as limitations, as many variations thereof are possible without departing from the spirit and scope of the invention, which would be recognized by one of ordinary skill in the art.

TABLE 1

| Material | | Ex. 1 | Ex. 2 | Ex. 3 | Ex. 4 |
|---|---|---|---|---|---|
| Name | INCI Name | As Added | | | |
| Panthenol | Panthenol | 0.20 | 0.70 | 0.70 | 0.70 |
| Water* | Water | 43.56 | 43.06 | 68.06 | 43.26 |
| Sepigel 305¹ | Polyacrylamide/C13-14 | 0.82 | 0.82 | 0.82 | 0.82 |
| | Isoparaffin/Laureth-7 | | | | |
| Amaze ™ XT² | Dehydroxanthan | 0.85 | 0.85 | 0.85 | 0.65 |
| | Gum | | | | |
| Caffeine | Caffeine | 0.94 | 0.94 | 0.94 | 0.94 |
| Niacinamide | Niacinamide | 3.13 | 3.13 | 3.13 | 3.13 |
| Sunny Citrus | Perfume | 0.50 | 0.50 | 0.50 | 0.50 |
| Ethanol | Alcohol | 50.00 | 50.00 | 25.00 | 50.00 |

pH adjusted to 5.5-6.5
viscosity adjusted to 5,000-20,000
*water is QS for pH and viscosity adjustments
¹Sourced from Seppic
²Sourced from AkzoNobel

TABLE 2

| Material | | Ex. 5 | Ex. 6 | Ex. 7 | Ex. 8 |
|---|---|---|---|---|---|
| Name | INCI Name | As Added | | | |
| Panthenol | Panthenol | 0.70 | 0.70 | 0.70 | 0.70 |
| Water* | Water | 47.33 | 46.39 | 44.20 | 41.66 |
| Sepigel 305¹ | Polyacrylamide/C13-14 | 0.82 | 0.82 | 0.82 | 2.22 |
| | Isoparaffin/Laureth-7 | | | | |
| Amaze ™ XT² | Dehydroxanthan | 0.65 | 0.65 | 0.65 | 0.85 |
| | Gum | | | | |
| Caffeine | Caffeine | 0.00 | 0.94 | 0.00 | 0.94 |
| Niacinamide | Niacinamide | 0.00 | 0.00 | 3.13 | 3.13 |
| Sunny Citrus | Perfume | 0.50 | 0.50 | 0.50 | 0.50 |
| Ethanol | Alcohol | 50.00 | 50.00 | 50.00 | 50.00 |

pH adjusted to 5.5-6.5
viscosity adjusted to 5,000-20,000
*water is QS for pH and viscosity adjustments
¹Sourced from Seppic
²Sourced from AkzoNobel

In the examples, all concentrations are listed as weight percent, unless otherwise specified and may exclude minor materials such as diluents, filler, and so forth. The listed formulations, therefore, comprise the listed components and any minor materials associated with such components. As is apparent to one of ordinary skill in the art, the selection of these minors will vary depending on the physical and chemical characteristics of the particular ingredients selected to make the hair care compositions.

TABLE 3
DATA

| Material | | |
|---|---|---|
| Name | INCI Name | As Added |
| Panthenol | Panthenol | 0.70 |
| Water* | Water | QS |
| Amaze ™ XT² | Dehydroxanthan | Varies |
| | Gum | |
| Sepigel 305¹ | Polyacrylamide/C13-14 | 0.82 |
| | Isoparaffin/Laureth-7 | |
| Caffeine | Caffeine | 0.94 |
| Niacinamide | Niacinamide | 3.13 |
| Sunny Citrus | Perfume | 0.50 |
| Ethanol | Alcohol | 50.00 |

*water is QS for pH and viscosity adjustments
¹Sourced from Seppic
²Sourced from AkzoNobel

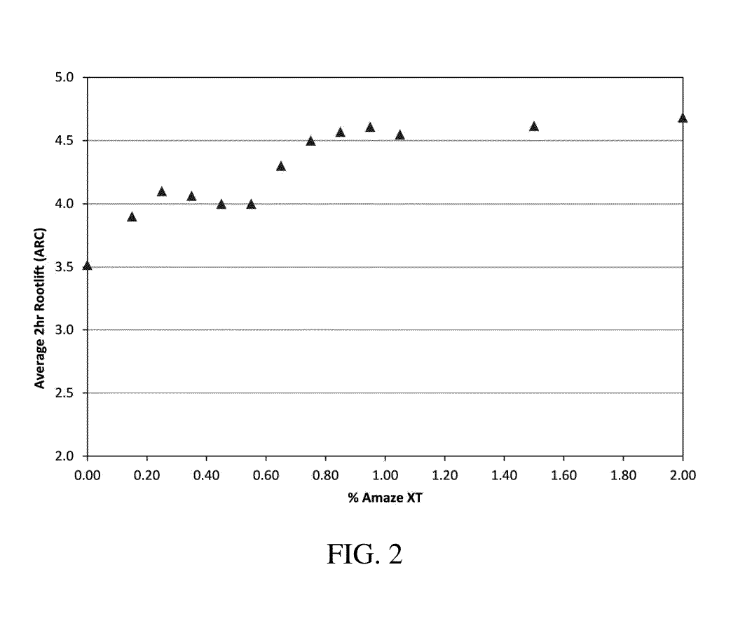

Table 4 lists different batches of the hair care composition that were made by varying the level of dehydroxanthan gum in the base formula found in Table 3. All batches were tested using the Root Lift Test Method described below. The “2 hr Average Root Lift” results are listed in Table 4. The “2 hr Average Root Lift” results are also graphed in FIG. 2.

TABLE 4

| | | Initial Viscosity | 2 hr Average Root |
|---|---|---|---|
| Number | Description | (cps)* | Lift |
| 1 | 0.00% Amaze | 637 | 3.5 |
| | XT | | |
| 2 | 0.15% Amaze | 1,667 | 3.9 |
| | XT | | |
| 3 | 0.25% Amaze | 3,792 | 4.1 |
| | XT | | |
| 4 | 0.35% Amaze | 7,736 | 4.1 |
| | XT | | |
| 5 | 0.45% Amaze | 15,894 | 4.0 |
| | XT | | |
| 6 | 0.55% Amaze | 19,088 | 4.0 |
| | XT | | |
| 7 | 0.65% Amaze | 22,988 | 4.3 |
| | XT | | |
| 8 | 0.75% Amaze | 29,290 | 4.5 |
| | XT | | |
| 9 | 0.85% Amaze | 33,939 | 4.6 |
| | XT | | |
| 10 | 0.95% Amaze | 37,091 | 4.6 |
| | XT | | |
| 11 | 1.05% Amaze | 40,287 | 4.5 |
| | XT | | |
| 12 | 1.50% Amaze | 60,172 | 4.6 |
| | XT | | |
| 13 | 2.00% Amaze | 80,129 | 4.7 |
| | XT | | |

TABLE 5

| Material Name | As Added |
|---|---|
| Alcohol | 50.00 |
| Water | 44.14 |
| Panthenol | 0.19 |
| Niacinamide | 3.13 |
| Caffeine | 0.94 |
| Abil B 8832 (Bis- | 0.30 |
| PEG/PPG-20/20 | |
| Dimethicone) | |
| Watergarden Craft 2 | 0.30 |

Table 6 shows the results of comparing different xanthan materials for root lift. Batches of a hair care composition were made by adding the different xanthan materials of Table 6 to the base formula of Table 5. All batches were tested for height average and ARC average using the Root Lift Test Method described below.

TABLE 6

| Material Added | Chemical Name | Height Average | ARC Average |
|---|---|---|---|
| VanZan | xanthan gum | 1.3 | 1.9 |
| Ultrapure | xanthan gum | 2.0 | 2.3 |
| Amaze XT | dehydroxanthan gum | 3.8 | 3.9 |

### Root Lift Test Method

Description:

The Root Lift “Short Switch” Test is a test that was developed to measure the maximum root lift provided by leave on treatment products. The test comprises of shorter 3″ switches which are treated, inverted, placed into the high humidity 80:80 room. Products are differentiated by hair fall and arc of hair switch (high humidity may cause both hair and some of the root lift benefit agents to plasticize, soften, and differentiate more quickly; typically, higher stiffness=better root lift).

### Test Equipment:

4 g 8″ switches cut to 3″ —general population hair switches

Root Lift “Short Switch” Test cart with 1″ grid behind 25 hair clamps (5 clamps/row)

Nikon camera

Black comb

Di-ionized water

Glass tub

Paper towels

Syringes

73:45 controlled temperature room

80:80 controlled temperature room

placement sheet

### Protocol:

A. N=3

B. Hair Preparation
1. Wet 4 g 4.5″ switch with di-ionized water.
2. Squeegee hair.
3. Paper towel blot hair to ensure it is dry.

C. Cart Preparation
1. Turn all hair claims to be vertical.
2. Ensure all metal rows are locked into cart columns.
3. Ensure 1″ grid is lined up straight with the metal rows.

D. Treatment
1. Treat switch with 0.2 mL at top (below tape) on both sides of the switch (0.4 mL per switch=0.25 ml/g of hair).
2. With gloves on, work product all the way through the switch.
3. Place switch in a hair clamp on the cart (randomizing treatments from clamp to clamp), hang straight down, make sure the switch is squeezed tightly so that the hairs are stuck together.
4. Record placement on the “Product Placement Sheet.”
5. Repeat steps 1-4 until 3 switches for each test product and control have been placed on the cart.
6. Store cart in a 73:45 room overnight.
7. Comb through switches—3× wide tooth, 3× fine tooth. Do one switch at a time. Make sure to place it back in its designated spot.
8. Move cart outside of the 80:80 room.
9. Invert all of the switches so that they are slightly tilted to the right hand side (if the hair falls the goal is to have them all fall in the same direction so that the arc measurements can be made correctly).
10. Place the cart in the 80:80 room and leave there for a designated time (typically do 2 hrs but can be adjusted for project needs).
11. At the end of each time point, in the 80:80 room, photograph each switch.
   a. Stand 12″ from switch.
   b. Ensure camera is level and not zoomed in.
12. Evaluate each switch for height and arc (see below).

### Data Analysis:

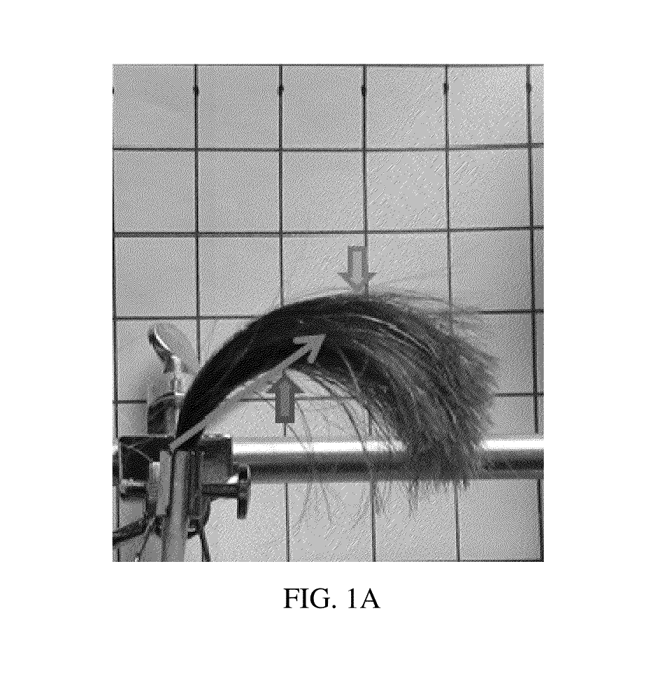

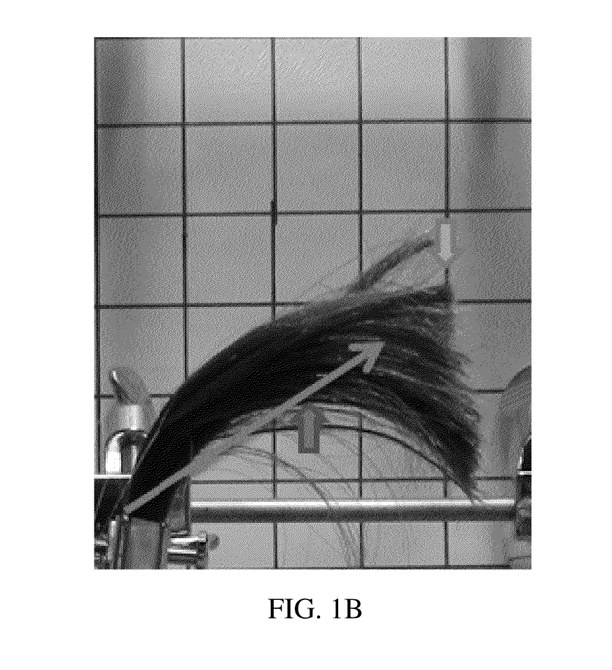

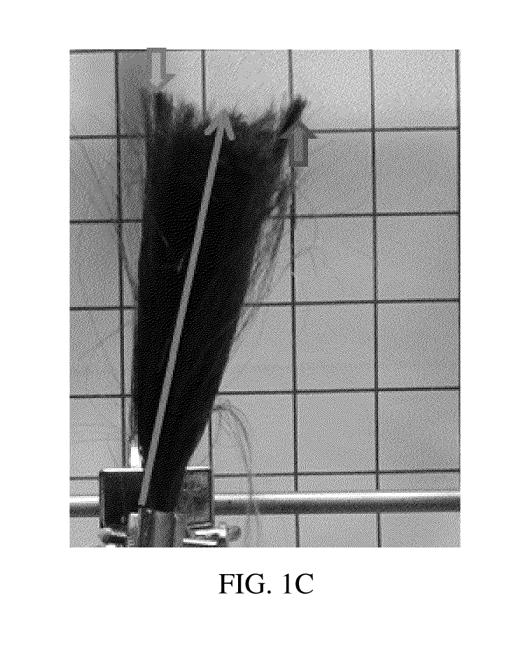

A. Background:
The switch of hair is superimposed onto an XY plot with the 1″ grid behind the hair clamps. This allows for various points on the switch to be assigned an XY value, estimating to a ½″. The maximum switch height and the arc length are calculated. As seen in FIG. 1A, the switch has curled over and generates the shortest arc metric (blue line [diagonal line]). As seen in FIG. 1B, the switch has only a slight increase in the maximum switch height over the switch in FIG. 1A but generates an arc metric almost twice as long. In this way, the increase in lift is captured as less switch curl is observed.

B. Measurements: (see FIG. 1A, FIG. 1B, and FIG. 1C)
1. Green arrow [down arrow]: Y axis=maximum height, X axis=maximum lift
2. Red arrow [up arrow]: Y axis=minimum height, X axis=minimum lift
3. Arc length (midpoint)=SQRT(Average(X)̂2+Average (Ys)̂2)

C. Examples:

| | Left - Y | Left - X | Right - Y | Right - X | Height | Arc |
|---|---|---|---|---|---|---|
| FIG. 1A | 2 | 2 | 1 | 1.5 | 2 | 2.3 |
| FIG. 1C | 4.5 | 0 | 4 | 2 | 4.5 | 4.4 |

### Viscosity Test Method

The viscosity of the hair care composition may be determined by a cone and plate viscometer/rheometer which measures the viscous drag resulting from the sample material contained in the gap between a rotating cone and a stationary plate. The geometry of the cone and plate may be such that the entire sample is subjected to a uniform shear rate. A Brookfield Rheometer RS, with Cone C75-1, 26.7° C. temperature, 2.5 mL sample size, at 2res/sec for 3 minutes may be used.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of Embodiments of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1) A hair care composition for providing improved root lift comprising:
a) at least 0.1% dehydroxanthan gum, by weight of the hair care composition;
b) from about 0.2% to about 1% of a panthenol compound, by weight of the hair care composition;
c) from about 0.1% to about 10% of a rheology modifier, by weight of the hair care composition; and
d) a carrier.

2) The hair care composition of claim 1, wherein the hair care composition comprises at least 0.6% of the dehydroxanthan gum, by weight of the hair care composition.

3) The hair care composition of claim 1, wherein the hair care composition comprises from about 0.3% to about 1% of the dehydroxanthan gum, by weight of the hair care composition.

4) The hair care composition of claim 1, wherein the hair care composition comprises from about 0.6% to about 1% of the dehydroxanthan gum, by weight of the hair care composition.

5) The hair care composition of claim 1, wherein the hair care composition comprises from about 0.7% to about 1% of the dehydroxanthan gum, by weight of the hair care composition.

6) The hair care composition of claim 1, wherein the hair care composition comprises from about 0.3% to about 0.8% of the panthenol compound, by weight of the hair care composition.

7) The hair care composition of claim 1, wherein the hair care composition comprises from about 0.2% to about 0.5% of the rheology modifier, by weight of the hair care composition.

8) The hair care composition of claim 1, wherein the hair care composition further comprises alcohol.

9) The hair care composition of claim 1, wherein the hair care composition further comprises from about 25% to about 50% alcohol, by weight of the hair care composition.

10) The hair care composition of claim 1, wherein the hair care composition further comprises caffeine.

11) The hair care composition of claim 1, wherein the hair care composition further comprises from about 0.01% to about 1% caffeine, by weight of the hair care composition.

12) The hair care composition of claim 1, wherein the hair care composition further comprises from about 0.65% to about 0.85% caffeine, by weight of the hair care composition.

13) The hair care composition of claim 1, wherein the hair care composition further comprises from about 0.7% to about 0.8% caffeine, by weight of the hair care composition.

14) The hair care composition of claim 1, wherein the hair care composition further comprises niacinimide.

15) The hair care composition of claim 1, wherein the hair care composition further comprises from about 0.01% to about 4% niacinimide, by weight of the hair care composition.

16) The hair care composition of claim 1, wherein the hair care composition further comprises from about 2.5% to about 3.5% niacinimide, by weight of the hair care composition.

17) A hair care composition for providing improved root lift comprising:
a) from about 0.3% to about 1% dehydroxanthan gum, by weight of the hair care composition;
b) from about 0.2% to about 1% a panthenol compound, by weight of the hair care composition;
c) from about 0.1% to about 10% of a rheology modifier, by weight of the hair care composition;
d) from about 25% to about 50% alcohol, by weight of the hair care composition;
e) from about 0.01% to about 1% caffeine, by weight of the hair care composition;
f) from about 0.01% to about 4% niacinimide, by weight of the hair care composition; and
g) water.
